(19) United States
(12) Patent Application Publication
(10) Pub. No.: US 2024/0245484 A1
(43) Pub. Date: Jul. 25, 2024

(54) VISUALIZATION SYSTEM WITH SPATIAL MEASUREMENTS

(71) Applicant: B. Braun New Ventures GmbH, Freiburg im Breisgau (DE)

(72) Inventors: Amir Sarvestani, Freiburg (DE); Jean Stawiaski, Kirchzarten (DE)

(21) Appl. No.: 18/419,039

(22) Filed: Jan. 22, 2024

(30) Foreign Application Priority Data
Jan. 23, 2023 (DE) 10 2023 101 578.8

(51) Int. Cl. A61B 90/00; A61B 34/20; A61B 50/13

(52) U.S. Cl. CPC A61B 90/06; A61B 34/20; A61B 50/13; A61B 90/361; A61B 90/37; A61B 2090/061; A61B 2090/371

(57) ABSTRACT

A surgical measuring system, method, computer-readable storage medium and computer program can be used for intraoperative spatial measurement and representation of geometric line segment lengths. The system includes a surgical visualisation unit, a navigation system, a visual display device, and a control unit. The control unit is adapted to sense a position of an instrument tip of a medical instrument and produce a digital overlay representation with the intracorporeal capturing. A virtual measuring device has a distance marking and/or a distance number corresponding to a real distance in the intracorporeal capturing and whose zero point forms the instrument tip. The virtual measuring device is arranged in parallel to the capturing plane, or a spatial end point is definable. The connection of both points forms a straight line that forms a virtual ruler. The control unit is adapted to output the overlay representation on the visual display device.

### CROSS-REFERENCE TO RELATED APPLICATION(S)

This application claims priority under 35 U.S.C. § 119 to German Application No. 10 2023 101 578.8, filed on Jan. 23, 2023, the content of which is incorporated by reference herein in its entirety.

### FIELD

The present disclosure relates to a surgical intraoperative measuring system for the up-to-date/real time intraoperative spatial measurement and representation of geometric line segment lengths or dimensions or distances, comprising: a surgical visualisation unit which is adapted to produce an intracorporeal capturing/imaging of an intervention region in a capturing/imaging plane, a navigation system which is adapted to track, by means of a tracking system, at least an instrument tip of a medical instrument or of medical devices (in the three-dimensional space relative to the patient) which is usable for the intervention in the intervention region so as to define a zero point for the measurement by means of the instrument tip, and a visual display device, particularly an OP monitor, for the visual output of a representation with at least the produced intracorporeal capturing. It may also be said that the disclosure relates to a surgical navigation system with an (integrated) intraoperative measuring system with corresponding features. In addition, the present disclosure relates to a measuring method and to a computer-readable storage medium as well as to a computer program.

### BACKGROUND

In neurosurgery the use of visualisation units and/or visualisation systems, for instance, a surgical microscope, and of navigation systems for a precise navigation of instruments or else the visualisation system itself has meanwhile become an increasingly important component of a surgical intervention in a patient.

The visualisation systems generate an enlarged view of the patient's tissue, but have the disadvantage that they do not enable any precise specific (geometric) measurements. Such measurements are, however, necessary to determine a size of anatomic structures of interest, such as, for instance, blood vessels, nerves, or tumours, and to assist the surgeon during an intervention.

For the precise measurement of lengths, dimensions, distances, or similar parameters, navigation systems may be used. These navigation systems according to the current state of the art offer measuring functions “on the fly”, i.e. a quick, up-to-date measurement, by indicating a virtual ruler directly at the tip of a navigated instrument. By means of this virtual ruler it is possible to carry out geometric measurements either along the instrument axis (for instance, a distance to a target tissue), or else exactly perpendicular to the instrument (for instance, diameter of a target). As a rule, virtual markings are indicated here which enable the user to perform the desired measurements intuitively. The virtual ruler is overlaid in the navigation capturings and/or images (for instance, CT capturings or MRT capturings) and output appropriately by means of a display device. In the case of a navigated visualisation system (for instance, a navigated surgical microscope) the ruler may also be placed and/or overlaid over the image of the visualisation system, such as, for instance, a microscope image. The images of the visualisation system are, however, always perpendicular to an optical axis of the visualisation system, i.e. to a surgical visual axis. Both in 2D capturings and in 3D capturings (images) the user thus always looks on a plane image which is perpendicular to his/her visual axis. If a navigated instrument, such as, for instance, a pointer/navigated pointer, is used, the instrument is, however, generally not perpendicular to the image plane and/or parallel to the optical axis and/or the visual axis, but forms the reference system for exactly the measurement. A virtual ruler is thus always aligned at the instrument itself, particularly in extension of the instrument axis. This has the disadvantage that spatial measurements with a measuring function of the type of ruler can thus generally not be carried out in parallel to the image plane. The user must try to hold the instrument as parallel as possible to the capturing plane or perpendicular thereto with a virtual measuring ruler perpendicular to the instrument axis. But even in this variant no exact measurement can be carried out since an inaccurate orientation of the instrument and of a position of an origin always exists. If the user intends, for instance, to measure the diameter of a blood vessel visible on the picture of the visualisation system, he/she cannot measure this parameter directly with a (navigated) virtual ruler.

An alternative to a navigated virtual ruler and the measurement (“on the fly”) would be a measuring function based on the definition of 3D points in space with a navigated instrument. By the selection of a point on one side of the blood vessel and a point on the other side it is, for instance, possible to determine the diameter of the blood vessel and to calculate and output it by means of the navigation system. However, such measurement requires predefined steps and interactions of the user, which makes the measurement much more time-consuming and distinctly less intuitive. Specifically, the user, particularly the surgeon, intraoperatively has to set the right points straight away, wherein any correction of these points distinctly increases the duration of an intervention, which is often already time-limited itself, so that a surgeon often refrains from such measurement and hence increases a risk for the patient.

Another new technology for intraoperative measurements for a surgical visualisation is based on that measurements are performed in 3D for virtual intraoperative measurements in laparoscopic and robot-aided operations. This system, however, does not offer an up-to-date “on-the-fly” measurement with a measuring function by means of a ruler which may be (virtually) connected or assigned to a single navigated instrument.

### SUMMARY

It is therefore an object of the present disclosure to avoid or at least mitigate the disadvantages of the state of the art and in particular to provide a surgical system, a method as well as a computer-readable storage medium and a computer program which provide, to medical professionals such as a surgeon, intraoperatively, i.e. during an operation, in a current live capturing at a predefinable position, appropriately and adequately, a virtual measuring device/a virtual measuring instrument which integrates real dimensions or distances or line segment lengths virtually in the intracorporeal capturing, namely independently of an orientation of a tracked instrument, such as a pointer. A further partial object may be to (equally) incorporate a virtual measuring instrument intuitively for a user in various modalities of view. Another partial object may be to provide a user with an option of a definition of an arrangement of a virtual distance indication so as to optionally incorporate a virtual distance indication independently of the alignment of the tracked instrument in space.

A basic idea of the present disclosure thus provides to enable an up-to-date measurement (“on-the-fly”) independently of the angularity or alignment of the navigated instrument used, and particularly to provide a virtual measuring device in parallel to the image plane and/or capturing plane of the visualisation system and/or the visualisation unit.

In other words, a measuring system is described which comprises the following components: a surgical visualisation system and/or visualisation unit (such as particularly a surgical microscope and/or an exoscope and/or an endoscope) for producing an intracorporeal (particularly up-to-date) capturing, a navigation system, and an instrument with an instrument tip which is tracked by the navigation system in the three-dimensional space (3D space), as well as a control unit (processing unit/processor unit), and a display device, particularly a display. The control unit is in particular specifically adapted, also preferably for calibration of the visualisation system, but particularly for tracking the position of the navigated instrument and for indicating its position in and/or on the intracorporeal capturing of the visualisation unit; and a specific adaption of the control unit for representing a virtual measuring device, particularly a virtual ruler in and/or on the intracorporeal capturing of the visualisation system, namely in parallel to the capturing plane or in the case of two defined points on a straight line between these two points (wherein one point forms the zero point).

It is thus an essential object of the present disclosure to provide a (geometric) spatial measuring system for the user by means of which he/she is capable of quickly, reliably, and intuitively measuring geometric distances of tissue on a live image and/or a live capturing (intracorporeal capturing) of the patient. A navigated instrument is used to select a point of interest in the surgical intervention region. A navigation system locates the 3D position of the tip of the instrument (in space, i.e. determines the X, Y, Z coordinates) and uses same as a zero point of a virtual measuring device, particularly a virtual ruler. A core aspect of the present disclosure thus consists in that the measurements may be carried out also in a plane parallel to the capturing plane of the visualisation system, and are consequently independent of the alignment of the instrument relative to the capturing plane and/or image plane. For this purpose, the instrument tip is first of all tracked in the 3D space (X, Y, Z position, particularly continuously or in the case of user input, for instance, discretely), and the instrument tip is indicated in the intracorporeal capturing (i.e. in the picture) of the visualisation system. Then, particularly a plane parallel to that of the imaging system is produced and connected with the tip of the instrument. In this plane it is possible to produce various kinds of virtual ruler forms and to overlay them over the intracorporeal capturing (the image) of the visualisation system. The virtual ruler may particularly be linear with markings at particular distances (similar to a usual school ruler). Alternatively or additionally, the virtual measuring instrument (similar to distance areas of a point in a topographic card-circular ruler) may be of annular configuration with circles which surround the tip of the instrument as an origin and/or zero point of the circle, wherein each circle defines a particular distance to the instrument tip, and are each particularly spaced apart equally from each other, for instance, every 5 mm in diameter. In the case of the linear ruler the user may particularly rotate the instrument, and the control unit may be adapted to sense the alignment of the instrument and to also rotate the virtual ruler along with this so as to carry out a measurement in a particular region. In the case of the circular ruler a rotation of the instrument is not necessary. In both cases no further interaction of the user is required to carry out a measurement, but merely the reading of the markings at the point concerned.

Also, pursuant to the one alternative the user may define two points with a navigated instrument, wherein both are 3D points (in space) which are sensed in the capturing/in the image of the visualisation system. After the defining of the second point, the measuring system indicates a ruler with the length between the two points and/or a scale with divisions. In this case, the measurement is generally not restricted to the image plane since the two points may be disposed in two different planes. A deviation from this would, for instance, be similar to a rubber band-like function, wherein, after the determination of the first point, a kind of rubber band with a scale between the fixed first point and the current tip of the instrument appears. This would enable to carry out measurements without defining a second point, wherein after the first definition of a starting point the instrument tip acts as a dynamic second point.

The measuring system preferably requires a calibrated visualisation system and/or visualisation unit, i.e. the image parameters are known with an external reference which is used for the tracking of the instrument. The external reference may for instance, be the housing of the visualisation system, for instance, the housing of the microscope head. Optionally, the housing of the visualisation system may be tracked by a navigation system. Alternatively or additionally, the intracorporeal capturing of the calibrated visualisation system may also be used to track the position of the instrument by means of machine vision.

Specifically, the surgical measuring system may comprise: a visualisation device and a navigation system, wherein the capturing of the visualisation system is preferably calibrated, a navigated and/or tracked instrument to define a starting point and/or zero point of a spatial measurement, wherein the capturing/the image of the visualisation system is overlaid with a virtual measuring device (particularly a virtual measuring ruler), wherein the orientation, particularly the pose, (structure) of the virtual measuring device, such as the ruler, is parallel to the capturing plane, particularly in the capturing plane, of the visualisation system, so that the capturing plane of the visualisation system is independent of the alignment of the instrument to the capturing plane of the visualisation device and the user may determine an end point of the spatial measurement by means of the form/structure of the virtual measuring device (for instance, ruler).

In other words, a surgical intraoperative measuring system is provided here for the up-to-date intraoperative spatial measurement and representation of geometric line segment lengths or dimensions or distances, comprising: a surgical visualisation unit adapted to produce an intracorporeal capturing of an intervention region in a capturing plane, a navigation system adapted to track, by means of a tracking system, at least an instrument tip of a medical instrument (in the three-dimensional (3D) space relative to the patient) which is usable for the intervention in the intervention region so as to define a zero point for the measurement by means of the instrument tip, and a visual display device, particularly an OP monitor, for the visual output of a representation with at least the intracorporeal capturing produced. The measuring system further comprises a control unit which is adapted: —preferably to calibrate the visualisation unit, —to detect, by means of the navigation system, at least one position of an instrument tip of the navigated instrument, particularly a pose of the instrument with the instrument tip, —to produce a digital overlay representation/image with at least the intracorporeal capturing and particularly at least one virtual reference representation of the instrument tip at the tracked position, particularly a point or circle as a reference representation of the instrument tip or a line with origin or zero point at the instrument tip along a longitudinal axis of the instrument during the sensing of a pose of the instrument, —to insert, in the overlay representation produced, further a virtual measuring device, particularly a virtual ruler, with at least one distance marking and/or distance number which corresponds to a real distance in the intracorporeal capturing and whose zero point forms/is the instrument tip, wherein the virtual measuring device: is arranged in parallel to the capturing plane, particularly lies in the capturing plane itself, to indicate a distance measurement in the capturing plane (so that the virtual measuring device is independent of the alignment of the instrument to the capturing plane), or, by means of the tracked instrument, a spatial end point is definable apart from the zero point, both of which form the straight line for the virtual measuring device in the form of a ruler, and —to output this overlay representation produced with at least the virtual measuring device by means of the display device.

The expression “capturing plane” means here a face which is orthogonal on an optical axis of the visualisation unit. For instance, in a 2D (two-dimensional) endoscope as a visualisation unit with a terminal optical system a two-dimensional image or video is taken, wherein the image and/or the video is perpendicular on the optical axis and thus defines the capturing plane.

The expression “virtual measuring device” or “virtual measuring instrument” means here a digital visual representation and incorporation of such a geometric virtual structure which enables a reading of a spatial distance and/or of a plurality of distances (directly in the intracorporeal capturing) and is shown in the predefined alignment quasi in a scaled manner. For instance, a virtual ruler may be incorporated and be scaled such that it indicates the correct and real dimensions for the capturing. If, for instance, a surgical microscope zooms optically or digitally into the tissue, the scaling of the virtual measuring device will also change correspondingly, so that always the actual distances are also indicated, similar to a CAD program or a world map with distance bars.

In particular, the navigation system can be a surgical computer tomography-based navigation system (CT-based) or a magnetic resonance imaging-based (MRI-based) navigation system that uses preoperative 3D images in the form of CT images or MRI images. In particular, the navigation system is an optics-based navigation system that uses a navigation camera to optically track instruments and optical markers spatially. Alternatively or additionally, the navigation system can be electromagnetic (EM) based and can have sensors for detecting an EM field. Preferably, the navigation system can have a preoperatively created and/or intraoperatively adaptable digital surgical plan, which provides annotations for 3D images, such as a trajectory, for example marked by an arrow, and/or a tissue designation, preferably with a tissue evaluation, and/or an intraoperative image. The navigation system is adapted to perform a registration of the patient.

In accordance with one embodiment the tracked instrument may comprise in the region of its instrument tip either a predefined optical pattern and/or a predefined optical shape which is stored in a storage unit, and the control unit may be adapted track, on the basis of the sensed optical pattern or the geometric shape, by means of the intracorporeal capturing of the surgical visualisation unit and by means of machine vision, the position of the instrument tip, particularly the pose of the instrument. In a classical navigation, preferably a rigid body may be fastened on the instrument. In image processing navigation the position of the instrument is located either with a known optical pattern at the tip of the instrument or without pattern by means of the known shape of the instrument. In both cases, however, the instrument tip has to be visible in the image of the visualisation system and/or the visualisation unit. In other words, the navigation system may be adapted to work by means of machine vision, wherein the navigation system is further adapted to use the (up-to-date live) capturing of the visualisation system to track an instrument, wherein the tracked instrument is particularly equipped either with a (known) predefined optical pattern and/or with a (known) predefined geometry.

In accordance with a further embodiment the measuring system may comprise a pointer and/or a suction tube and/or a surgical instrument as a tracked instrument. With those it is possible to define a position in space at least for the zero point. The tracked instrument may thus particularly be a surgical (navigation) pointer or a suction tube or a surgical instrument.

Specifically, the measuring system may comprise a surgical microscope and/or a surgical exoscope and/or a surgical endoscope as a visualisation system. Thus, the surgical measuring system, which comprises a 2D or 3D visualisation system such as an endoscope and/or an exoscope and/or a microscope in addition to a navigation system and a tracked instrument, may be used to carry out, independently of the angularity of the instrument, spatial measurements such as geometric distances parallel to the image plane of the visualisation system.

In accordance with a further embodiment the measuring system may further comprise a surgical robot to whose robot arm the visualisation unit is connected in a navigated manner. The visualisation system is thus preferably mounted on a robot arm. This makes it possible to particularly move and align the visualisation unit actively.

Preferably, the entire measuring system may be integrated in only one single mobile cart. In other words, the measuring system may be integrated as a module mobile on one single cart, so that the entire functionality is guaranteed with only one cart.

Preferably, the visualisation system and the navigation system may be integrated in one single medical (mobile) cart or may each be a part of two or more separate carts. For instance, the visualisation system may be integrated at and/or on a first cart and the navigation system on a separate, second cart.

In accordance with a further embodiment, in the overlay representation, a side-by-side representation of the intracorporeal capturing and additionally a navigation view may be produced, wherein the virtual measuring device in the intracorporeal capturing is shown additionally also in the correct pose in the navigation view so as to provide two viewing modalities with the same virtual measuring device for a user. Specifically, the virtual ruler may be indicated both on and/or in the navigation images and on and/or in the images of the visualisation system.

Preferably, the navigation system may comprise an external navigation camera which tracks the instrument with instrument tip.

In accordance with a preferred embodiment the measuring system may further comprise an input unit, the display device particularly may be configured in the form of a touch display, and the control unit may be adapted, by means of a user input of the input unit: to set a distance dimensioning of the virtual measuring device, particularly a distance dimensioning of 1 mm or 5 mm, and/or to set a geometric structure of the virtual measuring device, particularly in the form of a ruler line with distance dimensioning, or in the form of concentric circles whose radius increases in equal distances, and is positioned in the capturing plane. Specifically, the virtual ruler may be configurable by the user in the form of geometric increments or markings. Preferably, the virtual ruler may be configured by the user, particularly by means of an input unit such as, for instance, a touch display, in the form of different geometric structures such as lines or rings, all of which are disposed in the image plane of the visualisation system.

Specifically, the control unit may calculate a numerical distance between the zero point and the end point and insert this numerical distance as a distance number.

In accordance with a further embodiment the control unit may further be adapted to insert, in the overlay representation apart from the virtual measuring device a second virtual ruler in extension of a longitudinal axis of the instrument. In this manner, distance measurements and/or line segment indications in two different directions are possible.

The navigation system may particularly be an optical infrared navigation system and/or a navigation system by means of image processing and/or an electromagnetic navigation system. The navigation system may preferably comprise an external infrared-based camera.

Optionally, preoperative 3D images (particularly three-dimensional CT capturings or MRT capturings) may be imported by the control unit and be registered with the patient for a navigation. Furthermore, particularly a patient tracker/patient marker may be fastened on the patient which establishes a rigid relation (static transformation) to the patient.

With respect to a computer-implemented surgical intraoperative measuring method for the up-to-date intraoperative spatial measurement and representation of geometric line segment lengths or dimensions or distances the object is solved in that it comprises the steps of: preferably calibrating a visualisation unit which is adapted to produce an intracorporeal capturing of an intervention region in a capturing plane; sensing, by a navigation system which is adapted to track at least an instrument tip of a medical instrument (in the three-dimensional space relative to the patient) by means of a tracking system, at least one position of an instrument tip of the navigated instrument, particularly a pose of the instrument with instrument tip; producing, by a control unit, a digital overlay representation with at least the intracorporeal capturing as well as particularly a virtual reference representation of the instrument tip at the tracked position, particularly a point or circle as a reference representation of the instrument tip, further with a virtual measuring device, particularly a virtual ruler, with at least one distance marking and/or distance number which corresponds to a real distance in the intracorporeal capturing and whose zero point forms/is the instrument tip, wherein the virtual measuring device is arranged in parallel to the capturing plane, particularly lies in the capturing plane itself, so as to indicate a distance measurement in the capturing plane, or, by means of the tracked instrument, a spatial end point is defined apart from the zero point, both of which form the straight line for the virtual measuring device in the form of a ruler, and outputting this overlay representation produced with at least the virtual measuring device by means of a display device.

With respect to a computer-readable storage medium and also with respect to a computer program the objects are solved in that it comprises instructions which, when executed by a computer, cause same to perform the steps of the measuring method in accordance with the present disclosure.

### BRIEF DESCRIPTION OF THE DRAWINGS

The present disclosure will be explained in detail in the following by means of preferred embodiments with reference to the accompanying Figures.

FIG. 1 shows a perspective view of a surgical intraoperative measuring system in accordance with a preferred embodiment of the present disclosure.

FIG. 2 shows a view of an overlay representation of the surgical measuring system of FIG. 1 with a side-by-side representation of both a navigation view and an intracorporeal capturing, each with a virtual ruler shown in parallel to the capturing plane.

FIG. 3 shows a schematic perspective view of a virtual ruler with a coaxial arrangement of the ruler as well as an arrangement in the capturing plane for an understanding of the function.

FIG. 4 shows a further adjustable overlay representation of the surgical measuring system of FIG. 1 with a side-by-side representation of both a schematic navigation view and an intracorporeal capturing, each with a virtual measuring instrument shown in the form of concentric circles in parallel to the capturing plane.

FIG. 5 shows an overlay representation of the surgical measuring system of FIG. 1 with a side-by-side representation of both a schematic navigation view and an intracorporeal capturing, each with a virtual measuring instrument shown in the form of a virtual ruler between a first defined zero point in a first plane and a second defined end or support point in a second parallel plane.

FIG. 6 shows a schematic flowchart of a measuring method in accordance with a preferred embodiment of the present disclosure.

The Figures are of schematic nature and shall only serve for the understanding of the invention. Equal elements are provided with the same reference signs. The features of the various embodiments are interchangeable.

### DETAILED DESCRIPTION

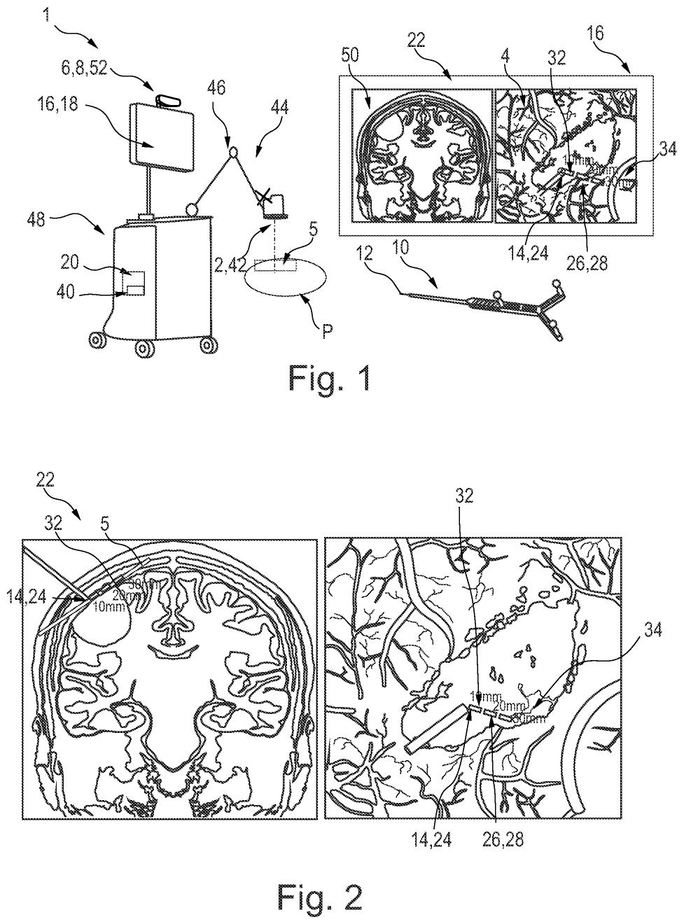

FIG. 1 shows in a perspective view a surgical intraoperative measuring system 1 (in the following only called measuring system) for the up-to-date intraoperative spatial measurement and representation of geometric line segment lengths or dimensions or distances. The measuring system 1 comprises a surgical visualisation unit 2 which is adapted to produce an intracorporeal capturing 4 of an intervention region of a patient P in a capturing plane 5 perpendicular to an optical axis. Furthermore, the measuring instrument comprises a navigation system 6 which is adapted to track, by means of a tracking system 8, at least an instrument tip 12 of a medical instrument 10 which is used for the intervention in the intervention region, so as to define a zero point 14 for the measurement by means of the instrument tip 12. The measuring system also has a visual display device 16, particularly an OP monitor 18, for the visual output of a representation with at least the intracorporeal capturing 4 produced.

In contrast to the state of the art, the measuring system 1 of the present disclosure comprises a specifically adapted control unit 20. It is configured to calibrate the visualisation unit 2, particularly to run through a stored calibration algorithm, and to then sense/detect a pose of the instrument 10 with position of the instrument tip 12 by means of the navigation system 6. The control unit 22 is further specifically configured to produce a digital overlay representation 22 both with the intracorporeal capturing 4 and with a virtual reference representation 24 of the instrument tip 12 at the tracked position, wherein here a circle has been chosen as a reference representation of the instrument tip. Furthermore, the control unit is adapted to additionally insert, in the overlay representation 22 produced, a virtual measuring device 26, particularly in the form of a virtual ruler 28 or of concentric (distance) circles 30 around the instrument tip, with distance markings 32 and a distance number (indication) 34 which correspond to the actual, real distance in the intracorporeal capturing 4. By means of the navigation and/or the navigation system 6 the control unit is always capable of calculating and incorporating the geometric relations. The zero point 14 of the virtual measuring device 26 forms the instrument tip 12. The virtual measuring device 26 is, however, arranged very specifically by the control unit 22 and inserted in the overlay representation 22, namely here either in a first mode in parallel to the capturing plane 5, particularly disposed in the capturing plane 5 itself, to indicate a distance measurement in the capturing plane 5, or that a spatial end point 36 is definable by means of the tracked instrument 10 apart from the zero point, wherein these defined points 14, 36 both form the straight line for the virtual measuring device 26 in the form of a virtual ruler 28. Finally, the control unit is adapted to output the produced overlay representation 22 with at least the virtual position of the instrument tip 12 and the virtual measuring device 26 by means of the display device 16. The patient P is indicated schematically only.

With the presently described and provided measuring system the surgeon can thus not only have a virtual ruler indicated in extension of an instrument 10, but can place a virtual ruler in the capturing plane 5 or between two points and have the correspondingly scaled distance dimensioning and/or line segment dimensioning (which corresponds to the real dimensioning) output there. The navigation system 6 serves as a basis for measuring the actual distances, similar to a CAD object.

FIG. 2 shows schematically an overlay representation 22 of the measuring system 1 of FIG. 1 with a virtual ruler 28 as a virtual measuring device 26 which is aligned in parallel to the capturing plane 5 of the visualisation system 2, wherein the reference point and/or zero point 14 is the 3D position (i.e. X, Y, Z coordinate) of the instrument tip 12 of the navigated instrument 10. In the overlay representation of FIG. 2, a navigation view 50 with the virtual instrument 10 and the virtual ruler 28 is shown on the left side, and in the right part of the side-by-side representation the up-to-date intracorporeal live capturing 4, also with the instrument 10 and the virtual ruler 28 shown. The virtual ruler 28 is aligned equal in relation to the patient P, so that the (same) virtual ruler 28 is indicated with the correct pose in the navigation view 50 and in a correlating manner with the correct pose in the intracorporeal capturing, wherein additionally the capturing plane 5 is shown in the navigation view 50 so as to directly display to the surgeon in which plane he/she is relative to the patient.

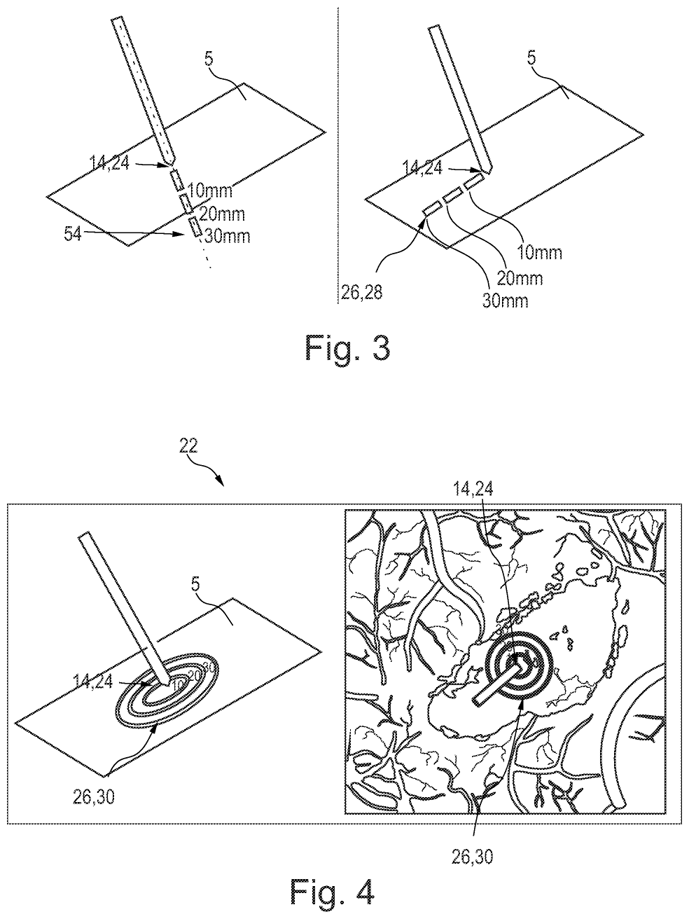

FIG. 3 shows a schematic representation of the virtual ruler 28 with a navigated instrument 10 and a capturing plane 5 of the visualisation system 2. In the left partial half a virtual ruler second virtual ruler 54 is aligned along a longitudinal axis of the instrument 10, and in the right partial half of FIG. 3 the virtual ruler 28 is aligned in parallel to the capturing plane 28. While the right partial half relates to an aspect of the present disclosure, the control unit 20 may further be adapted to additionally show a second virtual ruler 54 so as to offer the surgeon even further measuring possibilities if required.

FIG. 4 shows, instead of a line-based virtual ruler 28 of FIG. 2, an alternative overlay representation 22 with a structure of the virtual ruler using rings 30 and/or circles with a constant distance between the rings themselves which are concentric to the instrument tip 12. The circles and/or rings 30 are disposed in parallel to the capturing plane 5 of the visualisation system 2, namely independently of the alignment/orientation of the instrument 10, so that the physician may very easily read a dimension in any direction without rotating the instrument. Thus, distance rings in parallel to the capturing plane 5 are indicated at the instrument tip 12.

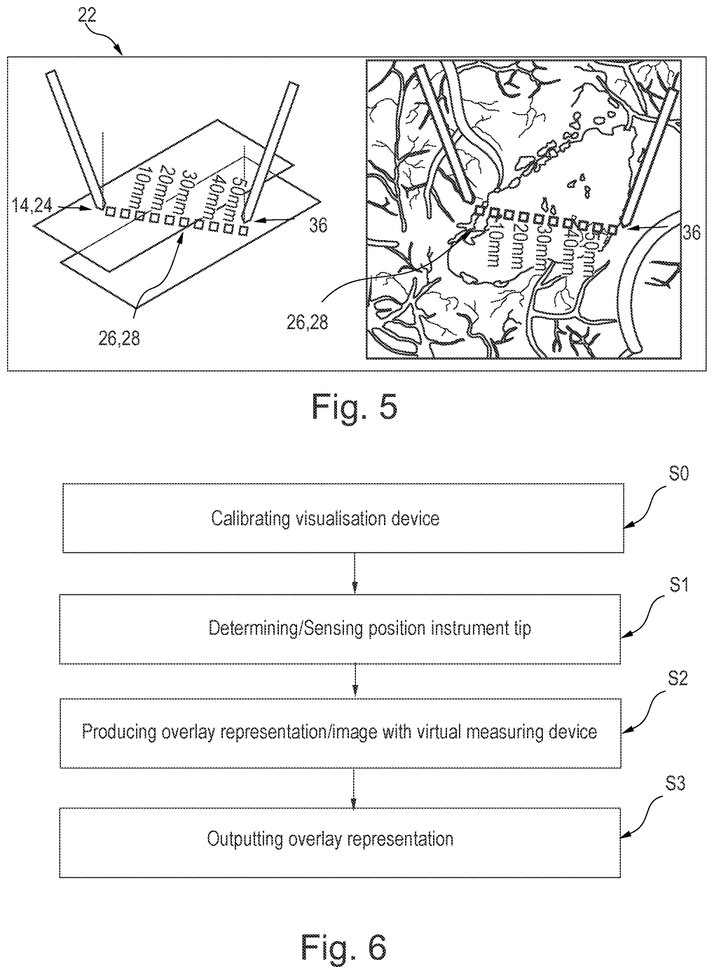

FIG. 5 shows an adjustable and/or user-selectable function alternative to the measurements in the capturing plane 5 in which the virtual ruler 28 can also be produced in two different planes based on two defined points, a zero point 14 and an end point 36. Also this alternative of the overlay representation 22 can be provided by the measuring system 1. The user thus defines at least a first zero point 14, particularly by means of the OP monitor in the form of a touch display, and has then the possibility of defining a second point, wherein these two set points 14, 36 then define a straight line and/or a line segment with the zero point 14, or else there is the option that the user, after definition of the zero pint 14, moves his/her tracked instrument 10 dynamically, and a virtual ruler 28 between the zero point 14 and the instrument tip 12 is always aligned and indicated up-to-date and dynamically.

FIG. 6 shows a flowchart of a computer-implemented surgical intraoperative measuring method for the up-to-date intraoperative spatial measurement and representation of geometric line segment lengths or dimensions or distances of a preferred embodiment of the present disclosure.

In a first optional step S0, preferably calibrating of a visualisation unit 2 may take place, which is adapted to produce an intracorporeal capturing 4 of an intervention region in a capturing plane 5.

In a step S1, sensing S1 by a navigation system 6 takes place, which is adapted to track, by means of a tracking system 8, at least an instrument tip 12 of a medical instrument 10, from at least one position of the instrument tip 12 of the navigated instrument 10.

In step S2, producing, by a control unit 20, of a digital overlay representation 22 with at least the intracorporeal capturing 4 as well as at least one virtual reference representation 24 of the instrument tip 12 at the tracked position takes place, particularly a point or circle as a reference representation of the instrument tip 12, further with a virtual measuring device 26, particularly a virtual ruler 28, with at least one distance marking 32 and/or distance number 34 which corresponds to a real distance in the intracorporeal capturing 4 and whose zero point forms/is the instrument tip, wherein the virtual measuring device 26 is arranged in parallel to the capturing plane 5, particularly is disposed in the capturing plane 5 itself, so as to indicate a distance measurement in the capturing plane 5, or a spatial end point 36 is defined apart from the zero point 14 by means of the tracked instrument 10, both of which form the straight line for the virtual measuring device 26 in the form of a virtual ruler 28.

In step S3, the outputting of this produced overlay representation 22 with at least the virtual reference representation 24 of the instrument tip 12 and the virtual measuring device 26 takes place by means of a visual display device 16.

1. A surgical intraoperative measuring system for up-to-date intraoperative measurement and representation of geometric line segment lengths or dimensions or distances in a surgical navigation, comprising:
a surgical visualization unit adapted to produce an intracorporeal capturing of an intervention region of a patient in a capturing plane;
a medical instrument comprising an instrument tip, the medical instrument being usable for an intervention in the intervention region so as to define a zero point for a measurement by means of the instrument tip;
a navigation system adapted to track, via a tracking system, at least the instrument tip of the medical instrument;
a visual display device for visually outputting a representation with at least the intracorporeal capturing; and
a control unit adapted to:
sense, by means of the navigation system, at least one position of the instrument tip of the medical instrument,
produce a digital overlay representation with at least the intracorporeal capturing,
insert, in the digital overlay representation, a virtual measuring device with at least one distance marking and/or distance number which corresponds to a real distance in the intracorporeal capturing with a zero point that forms the instrument tip, and
output the digital overlay representation with at least the virtual measuring device by means of the display device,
the virtual measuring device being arranged in parallel to the capturing plane to indicate a distance measurement in the capturing plane, or, by means of the medical instrument, a spatial end point is definable apart from the zero point, and the connection of both points forms a straight line for the virtual measuring device that forms a virtual ruler.

2. The surgical intraoperative measuring system according to claim 1, wherein the medical instrument comprises, in a region of the instrument tip, a predefined optical pattern and/or a predefined geometric shape stored in a storage unit for comparison, and wherein the control unit is adapted to track, based on the predefined optical pattern and/or the predefined geometric shape, the position of the instrument tip by means of the intracorporeal capturing of the visualisation unit and by means of machine vision.

3. The surgical intraoperative measuring system according to claim 1, wherein the medical instrument comprises a navigation pointer and/or a suction tube and/or a surgical instrument.

4. The surgical intraoperative measuring system according to claim 1, wherein the surgical visualization unit comprises a surgical microscope and/or a surgical exoscope and/or a surgical endoscope.

5. The surgical intraoperative measuring system according to claim 1, further comprising a surgical robot having a robot arm, wherein the surgical visualization unit is connected to the robot arm in a navigated manner and controllable by the control unit.

6. The surgical intraoperative measuring system according to claim 1, wherein the surgical intraoperative measuring system is integrated in or on one single mobile cart so as to provide a complete measuring system on the single mobile cart.

7. The surgical intraoperative measuring system according to claim 1, wherein a side-by-side representation of the intracorporeal capturing and a navigation view is produced in the digital overlay representation, and wherein the virtual measuring device is shown in the intracorporeal capturing and additionally in a correct pose in the navigation view so as to provide a user with two viewing modalities with the virtual measuring device.

8. The surgical intraoperative measuring system according to claim 1, further comprising an external navigation camera.

9. The surgical intraoperative measuring system according to claim 1, further comprising an input unit, wherein the control device is adapted, by means of a user input of the input unit to:
set a distance dimensioning of the virtual measuring device, and/or
set a geometric structure of the virtual measuring device.

10. The surgical intraoperative measuring system according to claim 1, wherein the control unit is adapted to calculate a numerical distance between the zero point and the end point and insert the numerical distance as a distance number.

11. The surgical intraoperative measuring system according to claim 1, wherein the control unit is further adapted to insert, in the digital overlay representation, a second virtual ruler in extension of a longitudinal axis of the medical instrument, apart from the virtual measuring device.

12. A computer-implemented surgical intraoperative measuring method for up-to-date intraoperative spatial measurement and representation of geometric line segment lengths or dimensions or distances, comprising the steps of:
producing an intracorporeal capturing of an intervention region in a capturing plane;
sensing, by a navigation system which is adapted to track at least an instrument tip of a medical instrument by means of a tracking system, at least one position of the instrument tip of the medical instrument;
producing, by a control unit, a digital overlay representation with at least the intracorporeal capturing of the intervention region in the capturing plane, further with a virtual measuring device, with at least one distance marking and/or distance number which corresponds to a real distance in the intracorporeal capturing and whose zero point forms the instrument tip, wherein the virtual measuring device is arranged in parallel to the capturing plane, so as to indicate a distance measurement in the capturing plane, or, by means of the medical instrument, a spatial end point is defined apart from the zero point, both of which form a straight line for the virtual measuring device in the form of a virtual ruler; and
outputting the digital overlay representation with a visual display device.

13. A computer-readable storage medium comprising instructions which, when executed by a computer, cause the computer to perform the steps of the computer-implemented surgical intraoperative measuring method according to claim 12.

14. A computer program comprising an instruction which, when executed by a computer, cause the computer to perform the steps of the computer-implemented surgical intraoperative measuring method according to claim 12.
